(19) United States
(12) Patent Application Publication
(10) Pub. No.: US 2024/0021288 A1
(43) Pub. Date: Jan. 18, 2024

(54) METHOD AND SYSTEM FOR PROVIDING A PROCEDURE PARAMETER

(71) Applicant: Siemens Healthcare GmbH, Erlangen (DE)

(72) Inventor: Marcus Pfister, Bubenreuth (DE)

(21) Appl. No.: 18/221,087

(22) Filed: Jul. 12, 2023

(30) Foreign Application Priority Data
Jul. 18, 2022 (DE) 10 2022 207 304.5

(51) Int. Cl. G16H 20/40; A61B 6/00

(52) U.S. Cl. CPC G16H 20/40; A61B 6/481

(57) ABSTRACT

Contrast agent administration is controlled for medical imaging. Anamnesis data of an examination object is received. A maximum value of a substance quantity is determined based on the anamnesis data. Planning information that has information with regard to a planned sequence of a procedure is received. The procedure includes an administration of substance. An instantaneous value of the substance quantity is acquired. A required value of the substance quantity for the planned procedure is determined based on the instantaneous value and the planning information. A procedure parameter is based on the required value and maximum value of the substance quantity. The procedure parameter has information with regard to a deviation or conformity between the required value and the maximum value of the substance quantity.

### RELATED APPLICATION

This application claims the benefit of German Application DE 10 2022 207 304.5, filed on Jul. 18, 2022, which is hereby incorporated by reference in its entirety.

### FIELD

The present document relates to a method for providing a procedure parameter, a system, and a computer program product.

### BACKGROUND

In the case of minimally invasive interventions, often therapies or diagnostic examinations are performed by medical objects, for example a diagnostic and/or therapeutic instrument, which are inserted at least in part into an examination object. Often, the medical objects are advanced via an access in a groin region of the examination object via guide wires and catheter under X-ray fluoroscopy control to a target position, in particular an operation site. A navigation into individual vascular outlets can be performed for example by rotation and/or translation, in particular advancement, of the guide wire. One example of this is the endovascular treatment of an abdominal aortic aneurysm (EVAR) by insertion of a stent graft.

In order to map vessels in X-ray fluoroscopy, an administration of contrast agent containing iodine is frequently required, for example in the case of angiography. On account of the kidney-damaging effect of iodine and since the examination objects often already have a limited kidney function, a use of iodine as a contrast agent should be limited to a necessary level. It is known from the publication by P. Steffens and N. Aichbauer, [“Kontrastmitteldosierung and Optimierungspotenzial in der CT”] “Contrast agent dosing and optimization potential in CT”, Radiopraxis, 13 (02), 2020, Pages 83-94 for example that in order to optimize a maximum contrast agent dose for contrast agent containing iodine, it is possible to draw upon patient-related factors, in particular an age and/or sex and/or weight and/or a size and/or existing underlying illnesses, for example diabetes, and/or kidney function parameters, for example an estimated glomerular filtration rate eGFR and/or a serum creatinine (sCr). In order to minimize a contrast agent quantity, there is moreover a method of fusion imaging, wherein pre- and intra-procedural mapping of the examination object can be superimposed in a positionally correct manner. In order to detect the pre- and intra-procedural state of the examination object, a plurality of angiography recordings is frequently required. Additional, in particular, unplanned angiographies, for example on account of emergencies, can disadvantageously lead to exceeding an iodine tolerance of the examination object.

Moreover, substances, for example anesthetics and/or anticoagulants, are frequently administered during medical procedures, for example surgical procedures and/or interventional procedures. In the event of emergencies, during medical procedures, it is possible for a tolerable substance quantity to be exceeded in an unplanned manner. Therefore, a minimization of the substance quantity to a required minimum is also of great importance in this case in order to prevent harm to the examination object.

### SUMMARY

It is therefore the object of the present disclosure to render possible an improved substance management in the case of medical procedures.

The object is achieved by the respective subject matter of the claims. Advantageous embodiments having expedient developments are the subject matter of the subordinate claims.

The present disclosure relates in a first aspect to an, in particular computer implemented, method for providing a procedure parameter. In an act a), anamnesis data of an examination object is received. In an act b), a maximum value of a substance quantity is determined based on the anamnesis data. In an act c), planning information is received that has information with regard to a planned sequence of a procedure. In this case, the procedure includes an administration of substance. In an act d), an instantaneous value of the substance quantity is acquired. In an act e), a required value of the substance quantity for the planned procedure is determined based on the instantaneous value and the planning information. In an act f), the procedure parameter is provided based on the required value and the maximum value of the substance quantity. In this case, the procedure parameter has information with regard to a deviation or conformity between the required value and the maximum value of the substance quantity.

Advantageously, the above-described method acts are performed at least in part one after the other and/or simultaneously.

Receiving the anamnesis data in act a) and/or receiving the planning information in act c) can include, in particular, acquiring and/or reading from a computer readable data storage device and/or receiving from a data storage unit, for example a database. Moreover, the anamnesis data and/or the planning information can be provided by a provision unit of a medical device, for example a medical imaging device. Alternatively, or in addition, the anamnesis data and/or the planning information can be acquired with the aid of a user input of a medical operator, in particular by an input unit.

The anamnesis data can have information with regard to the examination object. The examination object can be for example a human and/or animal patient. In particular, the anamnesis data can have information that characterizes an, in particular pre-procedural, state of the examination object. The anamnesis data can have for example information with regard to pre-existing conditions and/or intolerances, in particular allergies, and/or a medication and/or earlier medical procedures, in particular operations and/or examinations, and/or physiological parameters of the examination object, for example a size and/or a weight and/or an age of the examination object.

In the act b), the maximum value of the substance quantity is determined based on the anamnesis data. The substance quantity can describe a volume and/or a mass of substance. Moreover, the maximum value of the substance quantity can specify a maximum, in particular tolerable, volume and/or a maximum, in particular tolerable, mass of substance that can be present in the examination object, in particular can be injected into the examination object, taking into consideration the anamnesis data in a predetermined period of time. Advantageously, the predetermined period of time can include the planned procedure. The substance can include for example a medication, in particular an anesthetic and/or an anticoagulant, for example Heparin, and/or a contrast agent, in particular an imaging-visible, for example radiopaque, contrast agent.

The maximum value of the substance quantity can advantageously be determined with the aid of the anamnesis data, in particular with the aid of patient-related factors, in such a manner that an administration of the maximum value of the substance quantity within the predetermined period of time is tolerable for the examination object. The determination of the maximum value of the substance quantity based on the anamnesis data, in particular in dependence upon the anamnesis data, can be performed for example by a look-up table and/or by applying a function, in particular a trained function, to the anamnesis data.

The planning information can advantageously have information with regard to the planned, in particular chronological, sequence of the procedure. In particular, the planning information can have a list, in particular a sequence, of multiple acts of the planned procedure. The planned procedure, in particular at least one act of the planned procedure, in particular multiple acts of the planned procedure, can advantageously include an administration of substance. In this case, the planning information can advantageously have information with regard to minimum required and/or average, in particular usually actually, performed acts that include the administration of substance. This information can have been determined for example with the aid of a statistical evaluation of multiple monitored, in particular observed, procedures of this type. The planned procedure can moreover include further acts, for example an arrangement and/or movement of at least one medical object on or in the examination object that include at least in part no administration of substance. Moreover, the planning information can have information with regard to a substance quantity that is required during the planned procedure.

The acquisition of the instantaneous value of the substance quantity in act d) can include acquiring a user input of the medical operator and/or receiving a signal from an apparatus for the administration of substance. In this case, the instantaneous value of the substance quantity can quantify an accumulation of a volume and/or a mass of the substance in the examination object up to an acquisition point in time, in particular since the start of the planned procedure. In particular, the instantaneous value of the substance quantity can be determined by summing the volume and/or the mass of the substance up to the acquisition point in time. The instantaneous value of the substance quantity can quantify in particular an actual substance quantity that is present in the examination object at the acquisition point in time. The acquisition point in time can be arranged intra-procedurally, in particular during an at least partial implementation of the planned procedure.

In act e), a required value of the substance quantity for the planned procedure, in particular a remaining part of the planned procedure after the acquisition point in time in act d), can be determined based on the instantaneous value and the planning information. In this case, it is possible, with the aid of the planning information, for a continuation of the planned procedure to be determined for the acquisition point in time of the instantaneous value. In particular, in this case it is possible to determine which acts of the planned procedure have been performed at the acquisition point in time of the instantaneous value and which acts of the planned procedure have not yet been performed. The required value of the substance quantity can be determined with the aid of the planning information and based on the instantaneous value of the substance quantity, in particular assuming implementation of the remaining acts of the procedure in accordance with the plan. In this case, the required value can quantify a volume and/or a mass of the substance that are required for the procedure assuming that the remaining acts of the planned procedure after the acquisition point in time are performed in accordance with the plan. In this case, the required value can be different, in particular can be greater than or identical to the information with regard to the substance quantity, said information being included in the planning information and being required during the planned procedure.

The provision of the procedure parameter can include for example storing on a computer-readable storage medium and/or a display of a graphical representation of the procedure parameter by a representation unit and/or a transmission to a provision unit.

The provision of the procedure parameter can moreover include an identification of a deviation between the required value and the maximum value of the substance quantity, for example due to a comparison, in particular a determination of a difference and/or a quotient, between the required value and the maximum value of the substance quantity. In this case, it is possible to provide the procedure parameter having the information with regard to the identified deviation or with regard to a conformity between the required value and the maximum value of the substance quantity. The procedure parameter can have for example qualitative and/or quantitative information, for example the difference and/or the quotient between the required value and the maximum value of the substance quantity. Moreover, the procedure parameter can be time-resolved, in particular at least in accordance with the acts of the planned procedure, which remain after the acquisition point in time of the instantaneous value in accordance with the planning information.

The proposed embodiment can render possible an improved substance management in the case of medical procedures, in particular with the aid of the procedure parameter.

In a further advantageous embodiment of the proposed method, the substance can include a contrast agent. In this case, the procedure can include medical imaging having the administration of contrast agent, in particular the injection of contrast agent.

The medical imaging can include a recording of medical imaging data of the examination object by a medical imaging device, while the contrast agent is present in the examination object, for example an angiography. The medical imaging device can include for example a medical X-ray device, in particular a medical C-arm X-ray device, and/or a computed tomography system (CT system) and/or a magnetic resonance tomography system (MRT system) and/or an ultrasonic device and/or a positron emission tomography system (PET system). The contrast agent can advantageously be visible under imaging, for example X-ray radiopaque. For example, the contrast agent can contain iodine. Advantageously, the planning information can have information with regard to the planned procedure steps and the procedure steps include the medical imaging having the administration of contrast agent.

The proposed embodiment can render possible an improved contrast agent management in the case of medical procedures, including medical imaging having the administration of contrast agent, in particular with the aid of the procedure parameter.

In a further advantageous embodiment of the proposed method, the procedure parameter can moreover have an adaptation parameter for adapting the administration of substance. In this case, the administration of substance can be adapted based on the adaptation parameter in such a manner that the maximum value of the substance quantity is complied with.

The provision of the procedure parameter can include providing the adaptation parameter. The adaptation parameter can have a specification, for example a control command and/or an instruction and/or a workflow indication, for the adaptation of the administration of substance. The adaptation of the administration of substance can include for example an adaptation, in particular an increase or decrease, of a rate of the administration of substance and/or a volume and/or a mass of the substance that is to be injected in a predetermined period of time, and/or a concentration of the substance that is to be injected. Advantageously, the planning information has information with regard to the substance quantity that is required for the planned procedure, in particular a minimum and/or average value of the substance quantity that is required for the planned procedure. It is possible for the instantaneous value to be greater than the minimum and/or average value of the substance quantity on account of additional, in particular unplanned procedure acts having an administration of substance up to the acquisition point in time of the instantaneous value. Advantageously, the adaptation parameter can be determined based on the comparison between the required value and the maximum value of the substance quantity. In this case, the adaptation parameter can advantageously be determined in such a manner that the deviation between the required value and the maximum value is minimized. The adaptation parameter can moreover be time-resolved, in particular at least in accordance with the acts of the planned procedure, which remain after the acquisition point in time of the instantaneous value in accordance with the planning information. In this case, the adaptation parameter can have a time-resolved specification with regard to the adaptation of the administration of substance in the acts of the planned procedure, which remain after the acquisition point in time of the instantaneous value in accordance with the planning information. Advantageously, the administration of substance for the remaining acts of the procedure in accordance with the planning information can be adapted by the adaptation parameter in such a manner that an adapted required value of the substance quantity is lower than or identical to the maximum value of the substance quantity.

The workflow indication can include a specification, for example an instruction and/or a control command and/or a character sequence and/or a graphical element, for example a symbol, which has information regarding the adaptation of the administration of substance. In this case, the provision of the procedure parameter can include an output of the workflow indication, for example a display of a graphical representation of the workflow indication and/or an output of an acoustic and/or haptic signal. For example, the workflow indication can signal, in particular display, that the required value exceeds the maximum value. Moreover, the workflow indication, in particular the specification, can specify the adaptation of the administration of substance in such a manner that the deviation between the required value and the maximum value of the substance quantity is minimized, in particular that the required value does not exceed the maximum value of the substance quantity.

Advantageously, a difference value can be determined as the difference between the required value and the instantaneous value of the substance quantity. By comparing the difference value with the difference value of the substance quantity, it is advantageously possible to identify from which of the still remaining procedure acts an implementation in accordance with the plan is required in order to comply with the maximum value of the substance quantity. Advantageously, the workflow indication can be information in this regard.

In particular, the procedure parameter can be provided to an apparatus for the administration of substance and/or a medical imaging device. The procedure parameter, in particular the adaptation parameter, can advantageously be provided, in particular transmitted, to the apparatus for the administration of substance, in particular a substance injector, for example a contrast agent injector, and/or a substance machine, and/or the medical imaging device.

The apparatus for the administration of substance can be designed so as to administer, in particular to inject, the examination object with a predetermined substance quantity. In this case, the apparatus can moreover be designed so as to receive the procedure parameter, in particular the adaptation parameter. Moreover, the apparatus can be designed so as to adapt, in particular in an automated manner, the administration of substance in dependence upon the procedure parameter, in particular the adaptation parameter.

The medical imaging device can include for example a medical X-ray device, in particular a medical C-arm X-ray device, and/or a computed tomography system (CT system) and/or a magnetic resonance tomography system (MRT system) and/or an ultrasonic device and/or a positron emission tomography system (PET system). The medical imaging device can be designed so as to record medical imaging data having mapping of the examination object, in particular of the substance that is present in the examination object. Advantageously, the procedure parameter, in particular the adaptation parameter, can be provided to the medical imaging device. In this case, the medical imaging device can be designed so as to adapt at least one recording parameter for the recording of the medical imaging data in dependence upon the procedure parameter, in particular the adaptation parameter.

The proposed embodiment can advantageously render it possible to comply with the maximum value of the substance quantity due to the provision of the procedure parameter having the adaptation parameter.

In a further advantageous embodiment of the proposed method, the determination of the required value of the substance quantity can include an extrapolation of the instantaneous value of the substance quantity based on the planning information.

The required value of the substance quantity can be extrapolated with the aid of the planning information and based on the instantaneous value of the substance quantity, in particular assuming implementation of the remaining steps of the procedure in accordance with the plan. Alternatively, or in addition thereto, the instantaneous value of the substance quantity can be compared with the information with regard to the substance quantity, which is included in the planning information and is at least and/or on average required for the planned procedure, in accordance with the progress of the procedure, in particular the already performed steps of the procedure until the acquisition point in time. With the aid of the comparison, it is possible to identify a deviation, in particular a ratio and/or a difference and/or a quotient, between the instantaneous value and the minimum value and/or average value of the substance quantity at the acquisition point in time. In this case, the deviation between the instantaneous value and the minimum and/or average value of the substance quantity can characterize a deviating, in particular increased, substance consumption in the previously performed steps of the procedure. The required value of the substance quantity can be extrapolated with the aid of the planning information and based on the instantaneous value of the substance quantity, assuming an implementation of the remaining steps of the procedure in accordance with the substance consumption that is identified at the acquisition point in time.

The proposed embodiment can render possible an improved estimation of the substance consumption for the remaining procedure steps.

In a further advantageous embodiment of the proposed method, the planned sequence of the procedure can include multiple procedure steps. In this case, the planning information can have at least one planning value with regard to the substance quantity in the procedure steps that include the medical imaging having the administration of substance. Moreover, the determination of the required value of the substance quantity can be based on the at least one planning value of the substance quantity.

The planned, in particular chronological, sequence of the procedure can include multiple steps, in particular procedure steps, which can be performed at least in part one after the other and/or simultaneously. Moreover, the planning information can have the at least one planning value, in particular in each case a planning value, with regard to the substance quantity that is required in the procedure steps, including the administration of substance. The at least one planning value can have a minimum value and/or average value of the substance quantity, in particular in each case a minimum value and/or average value of the substance quantity, with regard to the procedure steps of the planned procedure, which procedure steps include the medical imaging having the administration of substance. The average value can be predetermined for example in accordance with a standard substance quantity for the planned procedure (standard operating procedure, SOP).

The determination of the required value of the substance quantity can be based in addition on the at least one planning value of the substance quantity. In particular, the required value can be determined, in particular extrapolated, based on the instantaneous value based on the at least one planning value of the substance quantity for the remaining steps of the planned procedure, in particular after the acquisition point in time of the instantaneous value.

The proposed embodiment can render it possible to more precisely determine the required value of the substance quantity.

In a further advantageous embodiment of the proposed method, the procedure parameter can moreover have information with regard to a deviation or conformity between the required value and the at least one planning value of the substance quantity.

Advantageously, the procedure parameter can have, in particular qualitative and/or quantitative, information with regard to the deviation or conformity between the required value and the at least one planning value of the substance quantity, for example an accumulation, in particular a summation, of the at least one planning value for the steps of the planned procedure, which are performed at the acquisition point in time of the instantaneous value. For example, the procedure parameter can have a difference and/or a quotient between the required value and the at least one planning value of the substance quantity for the steps of the planned procedure that are performed at the acquisition point in time of the instantaneous value.

As a consequence, a prompt identification of the deviation between the required value and the at least one planning value of the substance quantity, for example by the operator, can be rendered possible with the result that, if necessary, required adaptations of the administration of substance can be performed in a timely manner.

In a further advantageous embodiment of the proposed method, the extrapolation of the instantaneous value of the substance quantity in addition can be based on a deviation between the instantaneous value and the at least one planning value of the substance quantity.

Advantageously, it is possible to identify the deviation, in particular the difference and/or the quotient, between the instantaneous value and the at least one planning value, in particular an accumulation of the at least one planning value, for the procedure steps that are performed up to the acquisition point in time. In this case, the deviation between the instantaneous value and the accumulated, in particular summed, at least one planning value of the substance quantity can characterize a deviating, in particular increased, substance consumption in the previously performed steps of the procedure. The required value of the substance quantity can be extrapolated based on the instantaneous value of the substance quantity, assuming an implementation of the remaining procedure steps in accordance with the identified deviation based on the at least one planning value for the remaining procedure steps.

As a consequence, an improved determination of the required value of the substance quantity can be rendered possible.

In a further advantageous embodiment of the proposed method, the procedure parameter can have a residual value of the substance quantity, which is determined by a comparison of the maximum value and the instantaneous value of the substance quantity.

Advantageously, the residual value of the substance quantity can quantify a difference between the maximum value and the instantaneous value of the substance quantity. In this case, the residual value of the substance quantity can quantify the substance quantity, in particular a mass and/or a volume of the substance quantity, which remains after the acquisition point in time for the procedure steps that still remain while complying with the maximum value of the substance quantity. In particular, the residual value of the substance quantity can indicate a maximum substance quantity for the remaining procedure steps of the planned procedure, which can be tolerated by the examination object. Advantageously, the provision of the procedure parameter can include providing the residual value of the substance quantity, for example a display of a graphical representation of the residual value.

The proposed embodiment can advantageously render it possible to reliably comply with the maximum value of the substance quantity.

In a further advantageous embodiment of the proposed method, the anamnesis data can include laboratory values and/or pre-procedural examination information and/or information from further procedures with regard to the examination object.

The anamnesis data can include, in particular pre-procedural, laboratory values, for example a creatinine value, of the examination object. Alternatively, or in addition thereto, the anamnesis data can have pre-procedural examination information, for example information regarding the administration of substance within the scope of pre-procedural examinations and/or information from imaging procedures, in particular a size and/or character and/or morphology of kidneys of the examination object, and/or information regarding pre-existing conditions and/or intolerances and/or a medication of the examination object. Alternatively, or in addition thereto, the anamnesis data can have information from further procedures, for example surgical and/or interventional procedures on the examination object. In this case, the maximum value of the substance quantity can advantageously be determined based on the laboratory values and/or the pre-procedural examination information and/or the information from the further procedures with regard to the examination object.

The proposed embodiment can render it possible to determine the maximum value of the substance quantity in an improved, in particular more precise, manner.

In a further advantageous embodiment of the proposed method, the instantaneous value of the substance quantity can be recorded with the aid of a user input and/or can be provided by an apparatus for the administration of substance.

Advantageously, the user input of the medical operator can be recorded by an input unit. The input unit can record the user input as a haptic input, for example by a keyboard and/or a pointing device and/or an input display, and/or optical input, for example by an optical sensor for gesture identification, and/or as an acoustic input, for example by an acoustic sensor for voice recognition. In this case, the user input can have information with regard to the instantaneous value of the substance quantity. As a consequence, the instantaneous value of the substance quantity can be recorded in particular also in the case of a manual and/or semi-automated administration of substance.

Alternatively, or in addition thereto, the instantaneous value of the substance quantity can be provided by the apparatus for the administration of the substance, in particular the substance injector and/or the substance machine. For this purpose, the apparatus for the administration of substance can have a sensor for detecting the substance quantity that is already administered and/or for detecting a substance quantity that is available in the apparatus. As a consequence, a particularly precise acquisition of the instantaneous value of the substance quantity can be rendered possible.

In a further advantageous embodiment of the proposed method, the determination of the maximum value of the substance quantity and/or the determination of the required value of the substance quantity can be based on machine learning.

In particular, the maximum value of the substance quantity can be determined by applying a first trained function to first input data. In this case, the first input data can be based on the anamnesis data, in particular can include the anamnesis data. Moreover, the required value of the substance quantity can be determined by applying a second trained function to second input data. In this case, the second input data can be based on the instantaneous value of the substance quantity and the planning information, in particular can include the instantaneous value and the planning information.

The first and/or second trained function can be trained in each case by a method of machine learning. In particular, the first and/or second trained function can be a first and/or second neural network, in particular a convolutional neural network (CNN) or a network including a convolutional layer.

The first and/or second trained function in each case map input data to output data. In particular, the first trained function maps the first input data to first output data. Moreover, the second trained function can map the second input data to second output data. In this case, the first and/or second output data depend in particular furthermore on one or multiple parameters of the respective trained function. The one or the multiple parameters of the first and/or second trained function can be determined and/or adapted by training. The determination and/or adaptation of the one or multiple parameters of the first and/or second trained function can be based in particular on a pair of training input data and associated training output data, in particular comparison output data, wherein the first and/or second trained function can be applied to the training input data so as to generate training mapping data. In particular, the determination and/or the adaptation can be based on a comparison of the training mapping data and/or the training output data, in particular comparison output data. In general, a trainable function, in other words a function having one or multiple parameters that are not yet adapted, is also referred to as a trained function.

Other terms for trained functions are trained mapping rule, mapping rule having trained parameters, function having trained parameters, algorithm based on artificial intelligence, algorithm of machine learning. One example for a trained function is an artificial neural network, wherein the edge weights of the artificial neural network correspond to the parameters of the trained function. In lieu of the term “neural network” it is also possible to use the term “neural net”. In particular, a trained function can also be a deep artificial neural network (also referred to as deep neural network). A further example for a trained function is a “support vector machine”, furthermore it is also possible in particular to use other algorithms of machine learning as the trained function.

Advantageously, the first and/or second trained function, in particular the first and/or second neural network, in each case have an input layer and an output layer. In this case, the input layer of the first trained function can be designed so as to receive the first input data. Moreover, the input layer of the second trained function can be designed so as to receive the second input data. Moreover, the respective output layer can be designed so as to provide mapping data, in particular output data. Moreover, the input layers and/or the output layers can in each case include multiple channels, in particular neurons.

Advantageously, at least one parameter of the first trained function can be adapted based on a comparison of a training maximum value with a comparison maximum value of the substance quantity. The comparison maximum value of the substance quantity can have been determined for example with the aid of training anamnesis data. The determination of the comparison value of the substance quantity based on the training anamnesis data, in particular in dependence upon the training anamnesis data, can be performed for example by a look-up table and/or with the aid of patient-related factors that are included in the training anamnesis data.

Advantageously, a provision of the first trained function can include an adaptation of the at least one parameter of the first trained function in such a manner that a deviation between the training maximum value and the comparison maximum value of the substance quantity is minimized.

Moreover, at least one parameter of the second trained function can be adapted based on a comparison of a training required value with a comparison required value of the substance quantity. The comparison required value can be determined for example by an extrapolation of a training instantaneous value of the substance quantity and based on training planning information. Advantageously, a provision of the second trained function can include an adaptation of the at least one parameter of the second trained function in such a manner that a deviation between the training required value and the comparison required value of the substance quantity is minimized.

The proposed embodiment can render it possible to determine the maximum value and/or the required value of the substance quantity in a computationally efficient manner.

In a further advantageous embodiment of the proposed method, the acts d) to f) can be performed repeatedly until a termination condition occurs.

Advantageously, the acts d) to f) can be performed repeatedly in a predefined time interval, at the start of a respective procedure act and/or in dependence upon the planning information. In this case, the instantaneous value of the substance quantity can be acquired. Moreover, the required value of the substance quantity for the planned procedure can be determined based on the, in particular most recently acquired, instantaneous value and the planning information. Moreover, the procedure parameter can be provided based on the, in particular most recently determined, required value and the maximum value of the substance quantity.

Advantageously, the termination condition can specify a maximum number of repetitions for the repeated implementation of the acts d) to f). Alternatively, or in addition, the termination condition can occur with the implementation, in particular completion, of the last planned procedure act.

The proposed embodiment can render possible an improved monitoring of the compliance with the maximum value of the substance quantity.

The present disclosure relates in a second aspect to a system including a provision unit that is designed so as to perform a proposed method for providing a procedure parameter. In this case, the provision unit can include a computing unit (computer), a storage unit (memory) and/or an interface (e.g., input device, display, computer network interface). The provision unit can be designed so as to perform the proposed method for providing a procedure parameter in that the interface, the computing unit and/or the storage unit are designed so as to perform the corresponding method acts. In particular, the interface can be designed so as to perform the acts a), c), d) and/or f). Moreover, the computing unit and/or the storage unit can be designed so as to perform the remaining acts of the proposed method, in particular the acts b) and/or e).

The advantages of the proposed system correspond essentially to the advantages of the proposed method for providing a procedure parameter. In this case, mentioned features, advantages or alternative embodiments can likewise also be transferred to the other claimed subjects and vice versa.

In a further advantageous embodiment of the proposed system, the system can furthermore include an apparatus for the administration of substance (pump) and/or a medical imaging device. In this case, the provision unit can be designed so as to provide the procedure parameter to the apparatus for the administration of substance and/or the medical imaging device. Moreover, the apparatus for the administration of substance can be designed so as to adapt an administration of the substance in dependence upon the procedure parameter. Furthermore, the medical imaging device can be designed so as to adapt a recording of medical imaging data of the examination object in dependence upon the procedure parameter.

The present disclosure relates in a third aspect to a computer program product having a computer program that can be loaded directly into a storage device of a provision unit (processor), having program sections in order to perform all the acts of a proposed method for providing a procedure parameter if the program sections are performed by the provision unit.

The present disclosure can moreover relate to a non-transitory computer-readable storage medium on which program sections, which can be read and can be performed by the provision unit, are stored in order to perform all the acts of the method for providing a procedure parameter if the program sections are performed by the provision unit.

A largely software-based realization has the advantage that provision units that have already been used previously can be retrofitted in a simple manner by a software update in order to function in the manner in accordance with the invention. Such a computer program product in addition to the computer program can where necessary include additional components such as for example a documentation and/or additional components, and also hardware components such as for example hardware keys (dongles etc.) in order to use the software.

### BRIEF DESCRIPTION OF THE DRAWINGS

Exemplary embodiments are illustrated in the drawings and are further described below. The same reference characters are used for identical features in different figures. In the drawings:

FIGS. 1 and 2 show schematic illustrations of various embodiments of a method for providing a procedure parameter,

FIG. 3 shows a schematic illustration of graphical representations of a substance consumption and a remaining substance quantity, according to one embodiment, and

FIG. 4 shows a schematic illustration of a proposed system, according to an embodiment.

### DETAILED DESCRIPTION

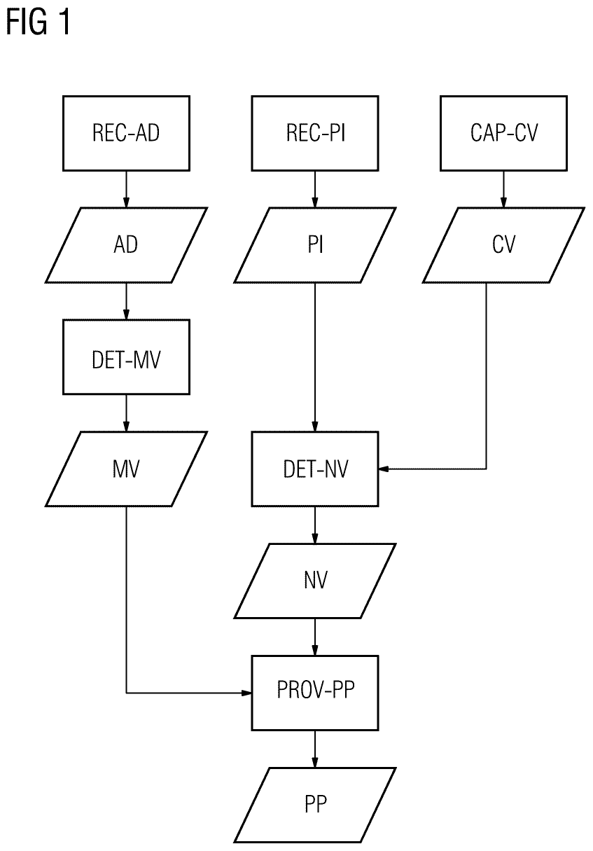

FIG. 1 illustrates schematically an advantageous embodiment of a proposed method for providing PROV-PP a procedure parameter PP. In this case, in a first act a) anamnesis data AD of an examination object can be received REC-AD. The anamnesis data AD can advantageously include laboratory values and/or pre-procedural examination information and/or information from further procedures with regard to the examination object. In a further act b), a maximum value MV of a substance quantity can be determined DET-MV based on the anamnesis data AD. In a further act c), planning information PI can be received REC-PI that has information with regard to a planned sequence of a procedure. In this case, the procedure can include an administration of substance. In particular, the substance can include a contrast agent. Moreover, the procedure can include medical imaging having the administration of contrast agent. In a further act d), an instantaneous value CV of the substance quantity can be acquired CAP-CV. The instantaneous value CV can be acquired for example with the aid of a user input and/or can be provided by an apparatus for the administration of substance. In a further act e), a required value NV of the substance quantity for the planned procedure can be determined DET-NV based on the instantaneous value CV and the planning information PI. In a further act f), the procedure parameter PP can be provided PROV-PP based on the required value NV and the maximum value MV of the substance quantity. In this case, the procedure parameter PP can have information with regard to a deviation or conformity between the required value NV and the maximum value MV of the substance quantity. Moreover, the procedure parameter PP can be provided PROV-PP to the apparatus for the administration of substance and/or a medical imaging device.

Advantageously, the procedure parameter PP can moreover have an adaptation parameter for the adaptation of the administration of substance. In this case, the administration of substance can be adapted based on the adaptation parameter in such a manner that the maximum value MV of the substance quantity is complied with.

The determination DET-NV of the required value NV of the substance quantity can advantageously include an extrapolation of the instantaneous value CV of the substance quantity based on the planning information PI.

The planned sequence of the procedure can include multiple procedure steps. In this case, the planning information PI can have at least one planning value of the substance quantity for the procedure steps that include the administration of substance, in particular the medical imaging having the administration of contrast agent. Moreover, the determination DET-NV of the required value NV of the substance quantity can be based on the at least one planning value of the substance quantity.

Advantageously, the procedure parameter PP can moreover have information with regard to a deviation or conformity between the required value NV and the at least one planning value of the substance quantity. In this case, the extrapolation of the instantaneous value CV of the substance quantity can be based in addition on a deviation between the instantaneous value CV and the at least one planning value of the substance quantity.

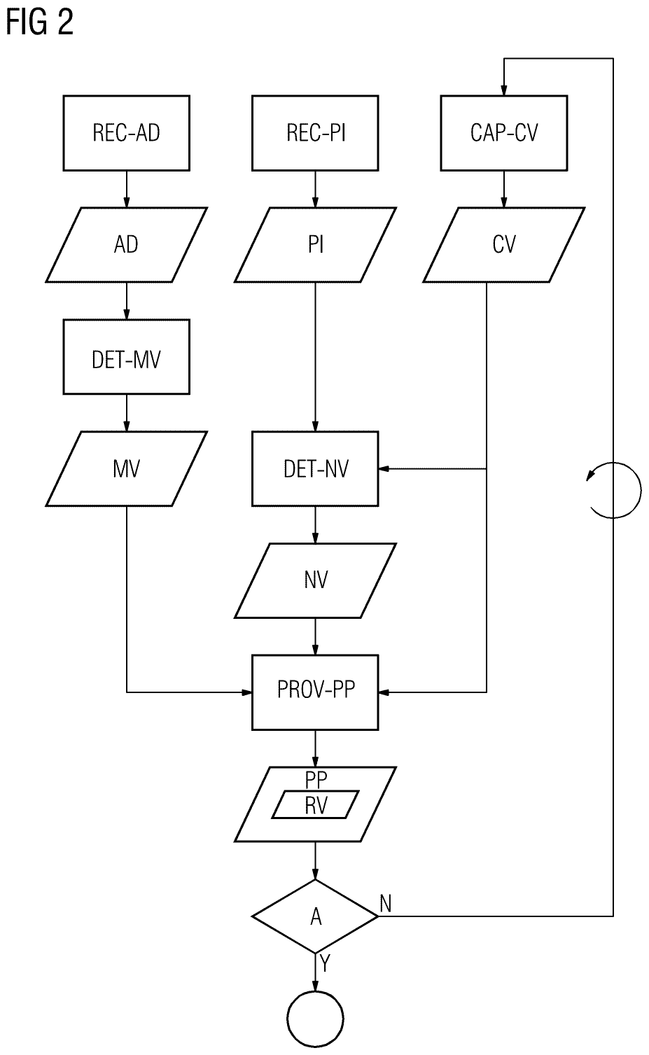

FIG. 2 illustrates a schematic illustration of a further advantageous embodiment of a proposed method for providing PROV-PP a procedure parameter PP. In this case, the acts d) to f) are repeatedly performed until the occurrence Y of a termination condition A. Moreover, the determination DET-MV of the maximum value MV and/or the determination DET-NV of the required value of the substance quantity can be based on machine learning.

Moreover, the procedure parameter PP can have a residual value RV of the substance quantity, which is determined by a comparison of the maximum value MV and the instantaneous value CV of the substance quantity.

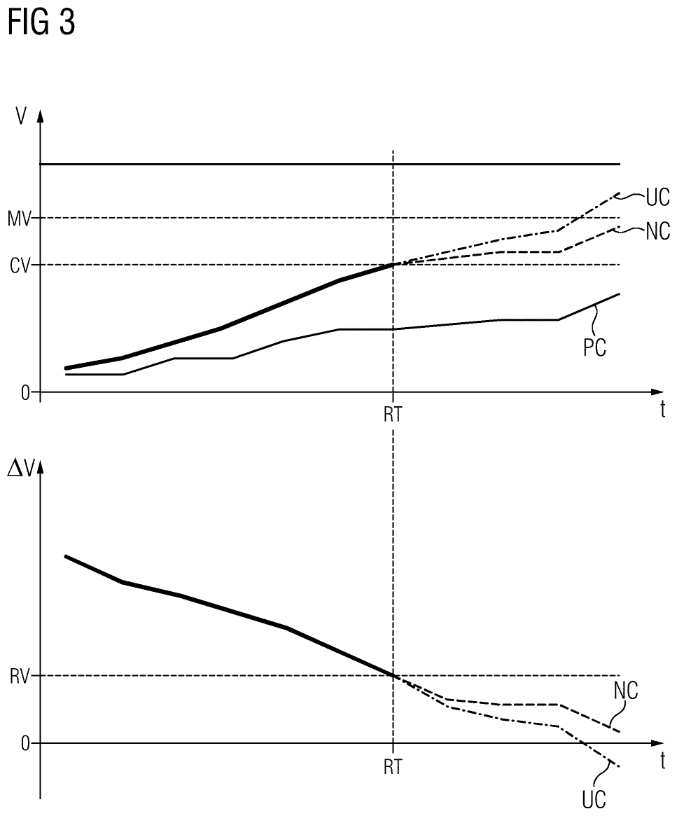

FIG. 3 schematically illustrates graphical representations of a substance consumption and a remaining substance quantity in each case in a diagram. In the upper diagram, an exemplary temporal curve of a consumption value V of the substance quantity during a medical procedure is illustrated schematically. The instantaneous value CV of the substance quantity can be acquired at an acquisition point in time RT. Moreover, three different extrapolated curves of the substance consumption are illustrated under different assumptions in the upper diagram. Under the assumption of an unamended administration of substance UC, it is possible to extrapolate a substance consumption that exceeds the maximum value MV of the substance quantity. Under the assumption of an implementation of the remaining procedure acts in accordance with the plan, in particular an administration of substance in accordance with the plan, it is possible to extrapolate the curve NC that ends in the illustrated example below the maximum value MV. Moreover, the entire curve PC of the substance consumption in accordance with the plan during the planned procedure is illustrated in the upper diagram. In this case, it is apparent that the instantaneous value CV of the substance quantity already exceeds the curve PC in accordance with the plan at the acquisition point in time RT. Advantageously, the curves UC and NC can be extrapolated in a time-resolved manner, in particular corresponding to at least the remaining planned procedure steps.

A difference AV between the consumption value V and the maximum value MV of the substance quantity is illustrated schematically in the lower diagram. At the acquisition point in time RT, it is possible by comparing the instantaneous value CV with the maximum value MV to determine the residual value RV of the substance quantity. Moreover, the curves, which are extrapolated from the residual value RV, of the remaining substance quantity, in particular available within the maximum value MV, under the assumption of the unamended administration of substance UC and the administration of substance NC in accordance with the plan for the remaining procedure acts are illustrated in the lower diagram. It is also apparent in this case that the remaining substance quantity is not sufficient in the event of the unamended administration of substance UC.

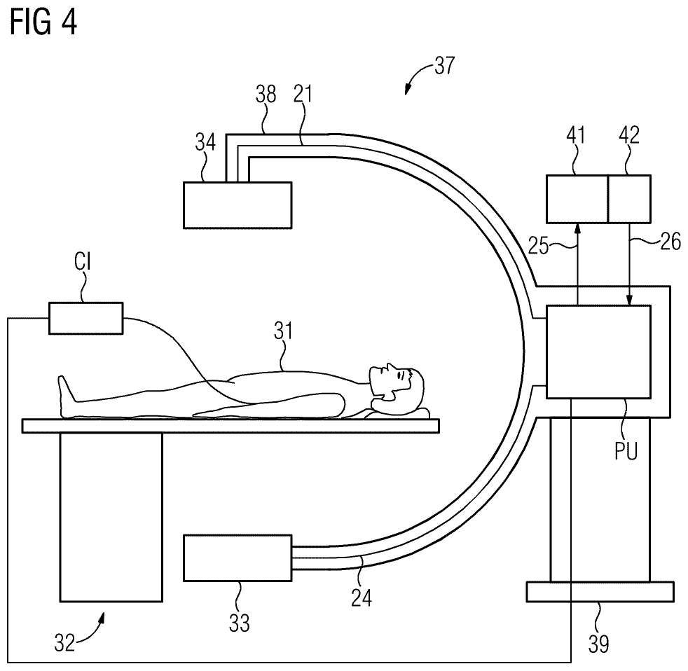

FIG. 4 illustrates schematically an advantageous embodiment of a proposed system. The system can include a provision unit PU, a representation unit 41, an apparatus for the administration of substance CI and a medical imaging device. In this case, the medical imaging device can include for example a medical C-arm X-ray device. The medical C-arm X-ray device 37 can be designed so as to record medical imaging data of the examination object 31 that is arranged on a patient positioning apparatus 32 and to provide said medical imaging data to the provision unit PU. Moreover, the apparatus for the administration of substance CI can be designed so as to inject a substance, in particular a contrast agent, into the examination object 31 prior to and/or during the recording of the medical imaging data. The medical C-arm X-ray device 37 can advantageously have a detector 34, in particular an X-ray detector, and a source 33, in particular an X-ray source, which are arranged on a C-arm 38 in defined arrangement with respect to one another. In order to record the medical imaging data of the examination object 31, the provision unit PU can transmit a signal 24 to the X-ray source 33. Thereupon the X-ray source 33 can emit an X-ray beam. When the X-ray beam impinges on a surface of the detector 34, after an interaction with the examination object 31, the detector 34 can transmit a signal 21 to the provision unit PU. The provision unit PU can receive the medical imaging data with the aid of the signal 21. In order to receive the medical imaging data, an arm 38 of the C-arm X-ray device 37 can be movably mounted about one or multiple axes. Moreover, the medical C-arm X-ray device 37 can include a movement apparatus 39 that renders possible a movement of the C-arm X-ray device 37 in the space.

The provision unit PU (processor) can moreover be designed (configured) so as to perform an embodiment of the proposed method for providing PROV-PP a procedure parameter PP. In this case, the provision unit PU can be designed so as to provide PROV-PP the procedure parameter PP to the apparatus for the administration of substance CI and/or the medical imaging device, in particular the medical C-arm X-ray device 37. The apparatus for the administration of substance CI can be designed so as to adapt the administration of the substance in dependence upon the procedure parameter PP. Moreover, the medical imaging device, in particular the medical C-arm X-ray device 37, can be designed so as to adapt the recording of the medical imaging data of the examination object 31 in dependence upon the procedure parameter PP.

The representation unit 41 can have or be for example a monitor and/or a display and/or a projector. The system can moreover include an input unit (input device) 42, for example a keyboard. The input unit 42 can preferably be integrated into the representation unit 41, for example in the case of a capacitive and/or resistive input display. The input unit 42 can be designed advantageously so as to detect a user input. The input unit can record the user input as a haptic input, for example by a keyboard and/or a pointing device and/or an input display, and/or optical input, for example by an optical sensor for gesture identification, and/or as an acoustic input, for example by an acoustic sensor for voice recognition. Moreover, the input unit 42 can transmit a signal 26 to the provision unit PU. The provision unit PU can be designed so as to control the medical C-arm X-ray device 37 in dependence upon the user input, in particular in dependence upon the signal 26. Moreover, the provision unit can be designed so as to acquire the instantaneous value CV of the substance quantity and/or the planning information PI, in particular the at least one planning value of the substance quantity, and/or the anamnesis data AD with the aid of the user input.

The representation unit 41 can be designed (configured) so as to display a graphical representation of the procedure parameter PP and/or the medical imaging data. For this purpose, the provision unit PU can transmit a signal 25 to the representation unit 41. In particular, the representation unit 41 can include a combined, in particular superimposed, graphical representation of the planning information PI, in particular the at least one planning value of the substance quantity, and/or the maximum value MV and/or the required value NV and/or the instantaneous value CV of the substance quantity. In this case, the graphical representation can be time-resolved, in particular at least in accordance with the planned procedure acts. Moreover, the combined graphical representation can include a graphical representation of the extrapolation of the instantaneous value CV of the substance quantity. Moreover, the graphical representation of the procedure parameter PP can include a graphical representation of the adaptation parameter and/or the workflow indication.

The schematic representations that are included in the described figures do not depict any scale or proportions.

Finally, reference is again made to the fact that the method and apparatuses that are described above in detail are only exemplary embodiments that can be modified in various ways by the person skilled in the art without departing from the scope of the invention. Furthermore, the use of the indefinite article “a” or “an” does not rule out that the relevant features can also be provided multiple times. Likewise, the terms “unit” and “element” do not rule out that the relevant components are made of multiple interacting part components that where necessary can also be spatially distributed.

1. A method for providing a procedure parameter, the method comprising:
a) receiving anamnesis data of an examination object,
b) determining a maximum value of a substance quantity based on the anamnesis data,
c) receiving planning information that has information with regard to a planned sequence of a procedure,
wherein the procedure comprises an administration of substance,
d) acquiring an instantaneous value of the substance quantity,
e) determining a required value of the substance quantity for the planned procedure based on the instantaneous value and the planning information, and
f) providing the procedure parameter based on the required value and maximum value of the substance quantity,
wherein the procedure parameter has information with regard to a deviation or conformity between the required value and the maximum value of the substance quantity.

2. The method as claimed in claim 1,
wherein the substance comprises a contrast agent, and
wherein the procedure comprises medical imaging having the administration of the contrast agent.

3. The method as claimed in claim 1,
wherein the procedure parameter comprises an adaptation parameter for the adaptation of the administration of substance,
wherein the administration of the substance adapts based on the adaptation parameter in such a manner that the maximum value of the substance quantity is complied with.

4. The method as claimed in claim 1,
wherein the determination of the required value of the substance quantity comprises an extrapolation of the instantaneous value of the substance quantity based on the planning information.

5. The method as claimed in claim 1,
wherein the planned sequence of the procedure comprises multiple procedure steps,
wherein the planning information has at least one planning value of the substance quantity in the procedure steps, which procedure steps comprise the administration of the substance, and
wherein the determination of the required value of the substance quantity is based on the at least one planning value of the substance quantity.

6. The method as claimed in claim 5,
wherein the procedure parameter moreover comprises information with regard to a deviation or conformity between the required value and the at least one planning value of the substance quantity.

7. The method as claimed in claim 5,
wherein the extrapolation of the instantaneous value of the substance quantity is based in addition on a deviation between the instantaneous value and the at least one planning value of the substance quantity.

8. The method as claimed in claim 1,
wherein the procedure parameter has a residual value of the substance quantity, which residual value is determined by a comparison of the maximum value and the instantaneous value of the substance quantity.

9. The method as claimed in claim 1,
wherein the anamnesis data comprises laboratory values, pre-procedural examination information, and/or information from further procedures with regard to the examination object.

10. The method as claimed in claim 1,
wherein the instantaneous value of the substance quantity is acquired with the aid of a user input and/or is provided by an apparatus for the administration of substance.

11. The method as claimed in claim 1,
wherein the determination of the maximum value of the substance quantity and/or the determination of the required value of the substance quantity is based on machine learning.

12. The method as claimed in claim 1,
wherein the acts d) to f) are repeatedly performed until the occurrence of a termination condition.

13. A system comprising:
a memory configured to store a computer program,
a processor configured, by the computer program, to:
a) receive anamnesis data of an examination object,
b) determine a maximum value of a substance quantity based on the anamnesis data,
c) receive planning information that has information with regard to a planned sequence of a procedure,
wherein the procedure comprises an administration of substance,
d) acquire an instantaneous value of the substance quantity,
e) determine a required value of the substance quantity for the planned procedure based on the instantaneous value and the planning information, and
f) provide the procedure parameter based on the required value and maximum value of the substance quantity,
wherein the procedure parameter has information with regard to a deviation or conformity between the required value and the maximum value of the substance quantity.

14. The system as claimed in claim 13, furthermore comprising an apparatus for the administration of the substance and/or a medical imaging device,
wherein the processor is configured so as to provide the procedure parameter to the apparatus for the administration of substance and/or the medical imaging device,
wherein the apparatus for the administration of substance is configured so as to adapt an administration of the substance in dependence upon the procedure parameter,
wherein the medical imaging device is designed so as to adapt a recording of medical imaging data of the examination object in dependence upon the procedure parameter.

15. A non-transitory computer readable storage medium having a computer program that, when executed by a computer, cause administration of a substance to a patient, the computer program including acts for:
a) receipt of anamnesis data of an examination object,
b) determination of a maximum value of a substance quantity based on the anamnesis data,
c) receipt of planning information that has information with regard to a planned sequence of a procedure,
wherein the procedure comprises an administration of substance,
d) acquisition of an instantaneous value of the substance quantity,
e) determination of a required value of the substance quantity for the planned procedure based on the instantaneous value and the planning information, and
f) provision of the procedure parameter based on the required value and maximum value of the substance quantity,
wherein the procedure parameter has information with regard to a deviation or conformity between the required value and the maximum value of the substance quantity.

16. The non-transitory computer readable storage medium of claim 15, wherein the determination of the required value of the substance quantity comprises an extrapolation of the instantaneous value of the substance quantity based on the planning information.

17. The non-transitory computer readable storage medium of claim 15, wherein the planned sequence of the procedure comprises multiple procedure steps,
wherein the planning information has at least one planning value of the substance quantity in the procedure steps, which procedure steps comprise the administration of the substance, and
wherein the determination of the required value of the substance quantity is based on the at least one planning value of the substance quantity.
